(19) United States
(12) Patent Application Publication
(10) Pub. No.: US 2008/0306458 A1
(43) Pub. Date: Dec. 11, 2008

(54) Surgical Irrigation and Retraction Platform

(75) Inventors: N.R. Chandrasekar, Canton, MA (US); Donald Mckay, Belmont, MA (US)

Correspondence Address: GOODWIN PROCTER LLP;PATENT ADMINISTRATOR, 53 STATE STREET, EXCHANGE PLACE, BOSTON, MA, 02109-2881, US

(73) Assignee: IQ Medical Devices, Belmont, MA (US)

(21) Appl. No.: 11/760,452

(22) Filed: Jun. 8, 2007

(51) Int. Cl. A61M 1/00; A61F 5/37

(52) U.S. Cl. 604/319; 128/846

(57) ABSTRACT

The invention relates to a universal design for an irrigation and retraction platform for use in operating on an extremity of a patient. The platform includes a chamber provided for the efficient collection and disposal of fluids to reduce the possibility of contaminating the surgical team and the operating room, and a support platform for positioning the extremity during surgery. The invention also includes an enclosure and other accessories for use with the platform.

### FIELD OF THE INVENTION

This invention relates generally to surgical platforms and enclosures, and more particularly to surgical platforms and enclosures including universal means for restraining and retracting an extremity of a patient and handling fluids used during irrigation of the extremity.

### BACKGROUND

When performing a surgical procedure on an extremity of a patient (e.g., a hand, forearm, or foot) there is a need to restrain and/or retract a portion of the extremity. Over the years, devices have been developed to position, for example, the hand and fingers for surgery. Except for the lead hand, which has been in use for many years, most have not been widely adopted. Lead hands, however, have a number of drawbacks. In use of the lead hand, the patient's fingers can slip out from under the lead fingers and exposure of the distal fingers is often blocked by the folded over end of the lead fingers. In addition, over time the lead hand can crack and develop sharp edges that can injure the patient and/or the surgeon. Towels are necessary to protect the hand from pressure with the inevitable shredding of lint. Additionally the lead hand being radio opaque means that it has to be removed whenever intra-operative X rays are needed.

Many of the operations in hand surgery deal with wounds that may not be heavily contaminated with foreign or infectious material. However, even clean surgical incisions or wounds may need intermittent irrigation during the procedure and before final closure to avoid desiccation of the tissues and removal of surgical debris. The volumes of fluid need not be large in these cases; however, lead hands and similar devices have no provisions for handling these fluids. The volumes of room temperature fluid needed for irrigation during a long case such as a microsurgical case are high and there is no efficient method of drainage of this fluid. It is also critical that the extremity not be moved during the procedure. Current platforms are limited in their design and function both in immobilization of the hand and forearm and collection of irrigation fluid.

Furthermore, there are a significant number of traumatic wounds involving extremities that are seen and treated by various specialists either in an emergency room setting or in an operating room. Regardless of the nature or etiology of the trauma, there can be a significant amount of contamination of these wounds. Whether the contamination is from solids or liquids or contamination from bacteria, in the case of infection, the wounds need to be thoroughly irrigated. This is accomplished by any one of several irrigation apparatus available at the present time. Most commonly, ER physicians use a simple syringe with a small shield, which is both very ineffective and harmful to tissues, in particular very delicate and fine tissues. In addition, the shield does not provide a fool proof seal against deflection of the irrigated sprays at high pressure. In general, the shields are not efficient, effective, or safe for either the patient or the ER physician. The problem of safely collecting the fluid remains.

In the operating room, several types of irrigators are available. These include battery or power assisted irrigators and bulb syringes, but none of these have proper shielding to protect the user. In some cases, bowls or basins are used to capture irrigant fluid; however, these basins are not specifically designed to handle even low volume irrigations safely. Frequently, either the basin, while in the operative field, accidentally tips over and spills contaminated fluid onto the field or the fluid is spilled from the basin in transferring the basin off the field. Typically, the extremity is held over a large basin that is placed next to the operative field or arm board while flushing is carried out. The extremity is then repositioned while often still wet over towels that may also be wet. Skin maceration may occur.

In addition, towels, lap pads, plastic plates, etc., which are also commonly used to capture irrigant fluids, are not effective at preventing accidents and fluid exposure. The towels, for example, can also worsen the situation by contaminating the wound with lint and/or other debris. In addition more assisting hands are needed. It is essential to have an efficient, safe, practical, and user friendly system for protecting the surgeon and other medical personnel, especially in contaminated or septic cases.

In extensively contaminated or infected wounds, an additional level of both protection and irrigation is needed, especially with the use of pulsed jet lavage. Current surgical platforms typically are not designed to be used with any type of enclosure to contain the fluids used in, for example, pulsed jet lavage. Jet lavage systems are capable of delivering high volumes of irrigating fluid to the affected area; however, there is no system for efficiently collecting and disposing of the fluid used. Also, neither the surgeon nor the assistants are protected from splashes, spills, or airborne particle contamination.

There is, therefore, a need for a device that both holds the patient's extremity in a position necessary for surgery combined with a platform for irrigation and efficient removal of the fluid. In addition, there is a need for an enclosure to assist in the irrigation of the extremity where large volumes of fluid are needed for irrigation.

### SUMMARY

The present invention is generally directed to a universal design for an irrigation retraction platform designed to fill a need in, for example, hand surgery. There are many cases in hand surgery where operative field irrigation is needed. This is true both of cases involving trauma, where there are open contaminated wounds, as well as long procedures where desiccation of tissue may occur. This is especially true in microsurgery. Also, an efficient collection and disposal system of fluids reduces the possibility of contamination of the surgical team and the operating room itself by spillage or splashing. Moreover, the use of towels and sponges to collect the liquids may be markedly reduced or eliminated. In addition, an easily used forearm, hand, and finger holder is provided for positioning of the extremity during surgery. The extremity can be well secured in any position or lie per the surgeon's choice or as required for the procedure. In addition, pegs and/or hooks can be used for secure holding of retraction sutures.

In addition, the invention is directed to an irrigation enclosure that can be easily fitted over the irrigation retraction platform to give an additional level of protection. In cases of severe infection or trauma with foreign material contamination of the upper or lower extremities, the enclosure includes provisions for thorough wound cleansing. At present, this is typically accomplished using bulb syringes or power assisted irrigators, including jet lavage systems. A platform and enclosure in accordance with the invention is substantially watertight, and in some designs, completely watertight, thus preventing the spread of the infected materials and/or contamination into the surrounding area (i.e., the rest of the operating room or emergency room), eliminates any splashing or spilling of the irrigation fluid by an efficient collection system that would not leak or become blocked, and makes the treatment of infection or contamination by irrigation thorough, safe, and effective.

In one aspect, the invention relates to an apparatus for supporting and restraining an extremity of a patient (e.g., a hand) during surgery. The apparatus includes a chamber having a bottom surface and four side walls, an opening defined by at least one of the bottom surface and sidewalls, a support platform disposed above the bottom surface and configured to engage at least a portion of the chamber, and a plurality of longitudinal perforations disposed through the support platform and arranged across the support platform, wherein the longitudinal perforations vary in size. In addition, the apparatus can include a plurality of restraints for interacting with pairs of correspondingly sized longitudinal perforations.

In various embodiments of the foregoing aspect, the plurality of longitudinal perforations includes a first portion of perforations arranged in a distal portion of the platform and oriented and sized to receive multiple restraints for a portion of the extremity, for example, a finger. The first portion of perforations can be arranged in a radial pattern and can be sized and arranged to provide multiple points of restraint for each finger. The plurality of longitudinal perforations can also include a second portion of perforations arranged in a medial portion of the platform and oriented and sized to receive additional restraints, such as, for example, at least one wrist restraint, and a third portion of perforations arranged in a proximal portion of the platform and oriented and sized to receive, for example, at least one forearm restraint. In one embodiment, the support platform is substantially planar. In one embodiment, the apparatus can include one or more suture retraction pegs disposed along a periphery of the chamber and/or the support platform. Optionally, the apparatus can include a positioning pad releasably disposable on the support platform. The pad can be positioned to support flexion of a portion of the extremity.

In additional embodiments, the apparatus includes a mesh layer disposed on the support platform and covering at least a portion of the plurality of perforations. The apparatus can also include a securing mechanism for securing the apparatus to a surgical or operating room table. The securing mechanism can be disposed on the chamber and/or the support platform and can include a clamp, a fastener, and/or an adhesive. In one embodiment, the apparatus includes a pedestal extending downwardly from the chamber and configured to support the apparatus at a working height. The height of the pedestal is adjustable. Additionally, the chamber can include a drip tray disposed non-parallel to the bottom surface and having at least one of an opening extending through a surface of the drip tray or a channel formed in the surface of the drip tray to facilitate drainage.

Furthermore, the plurality of restraints can include a first set of restraints sized for variably securing a finger and at least one second restraint sized for variably securing a wrist. Each restraint can include an elongate strap biased into a planar orientation, a plurality of projections arranged on a first end of the strap, and a second corresponding plurality of projections arranged on a second end of the strap. The apparatus can also include a gel pad disposed over at least a portion of the support platform to support the patient's forearm, wrist, hand, and/or fingers. The support platform can include at least one opening sized to pass fluid therethrough to the chamber. In one embodiment, the at least one opening includes a mesh. The support platform can include a plurality of holes sized and arranged to receive at least one thumb restraint, where the thumb restraint includes an elongate body including a proximal end configured for securely engaging at least one hole of the plurality of holes, a flange disposed about the elongate body adjacent the proximal end, and a loop disposed at a distal end of the elongate body. The loop is configured for receiving a thumb. The plurality of holes can include a first portion of holes disposed on a left medial side of the support platform and a second portion of holes disposed on a right medial side of the support platform. The apparatus can further include a support block having an inclined top surface and at least two projections extending downwardly from a bottom surface of the support block. The at least two projections can be configured for securely engaging at least two holes of the plurality of holes.

In additional embodiments, the apparatus includes an enclosure configured for sealingly engaging at least one of the chamber and the support platform. The enclosure can include a first opening disposed on one end of the enclosure and configured for receiving at least a portion of the extremity, for example, one of a patient's hand, wrist, forearm, or foot, and at least one second opening disposed on a side of the enclosure and configured for receiving at least one of a hand, a wrist, a forearm, and a surgical instrument. In addition, the enclosure can include a port disposed on a top surface thereof and configured for receiving a surgical instrument. In one embodiment, the enclosure is transparent.

In another aspect, the invention relates to an apparatus for assisting in performing a surgical procedure on an extremity of a patient. The apparatus can include a support platform for at least one of supporting and restraining at least a portion of the extremity, for example, a patient's hand, wrist, and/or forearm, and an enclosure configured to sealingly engage the support platform. The enclosure can include a first opening disposed on one end of the enclosure and configured for receiving at least a portion of the extremity and at least one second opening disposed on a side of the enclosure and configured for receiving at least one of a hand, a wrist, a forearm, and a surgical instrument. The enclosure can also include a port disposed on a top surface thereof and configured for receiving a surgical instrument, such as a jet lavage wand.

In another aspect, the invention relates to a surgical table, for example a hand table. The table includes a platform having a substantially planar top surface and defining a recess configured to receive an irrigation platform. The recess is sized and configured to position a work surface of the irrigation platform substantially level with the top surface. The table also includes a mechanism for securing the platform to an operating table.

In various embodiments of the table, the recess is sized and configured to allow horizontal positioning of the irrigation platform relative to the operating table. For example, the recess can be sized and configured to allow the platform to be moved towards or away from the operating table to accommodate varying arm lengths. At least a portion of the table, for example the platform, can be radio-lucent to facilitate the use of, for example, a C-arm type X-ray machine. In addition, the table can include one or more support legs. The support leg(s) can be foldable and its height can be adjustable.

These and other objects, along with the advantages and features of the present invention herein disclosed, will become apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic perspective top view of an apparatus in accordance with one embodiment of the invention;

FIG. 2A is a schematic plan view of the apparatus of FIG. 1;

FIG. 2B is a schematic side view of the apparatus of FIG. 1;

FIG. 2C is an enlarged view of a portion of the apparatus of FIG. 2B in accordance with an alternative embodiment of the invention;

FIG. 2D is a schematic cross-sectional view of the apparatus of FIG. 1 taken at line 2D-2D in FIG. 2A;

FIG. 2E is a schematic exploded view of the apparatus of FIG. 1;

FIG. 3A is a schematic plan view of an apparatus in accordance with an alternative embodiment of the invention;

FIG. 3B is a schematic cross-sectional view of the apparatus of FIG. 3A taken at line 3B-3B of FIG. 3A;

FIG. 4A is a schematic plan view of a restraint for use with the apparatus in accordance with one embodiment of the invention;

FIG. 4B is a schematic side view of the restraint of FIG. 4A;

FIG. 4C is a schematic side view of the restraint of FIG. 4A in use;

FIG. 5 is a schematic perspective view of a restraint in accordance with an alternative embodiment of the invention;

FIGS. 6A and 6B are schematic side and end views of a support block in accordance with one embodiment of the invention;

FIGS. 7A and 7B are front and side views of a suture retraction peg in accordance with one embodiment of the invention;

FIG. 7C is a partial side view of the platform of FIG. 1 illustrating alternative suture retraction pegs in accordance with one embodiment of the invention;

FIGS. 8A-8C are front, side, and top views, respectively, of a positioning pad in accordance with one embodiment of the invention;

FIG. 9A is a schematic perspective view of an enclosed apparatus in accordance with one embodiment of the invention;

FIGS. 9B-9D are enlarged cross-sectional schematic views of a portion of the apparatus of FIG. 9A illustrating alternative means of securing the enclosed apparatus;

FIGS. 9E-9G are schematic side, top, and end views, respectively, of the apparatus of FIG. 9A;

FIG. 9H is a schematic side view of the apparatus of FIG. 9A in accordance with an alternative embodiment of the invention;

FIG. 10 is a schematic perspective view of an apparatus in accordance with an alternative embodiment of the invention;

FIGS. 11A-11D are schematic top, end, and side views, respectively, of a surgical table in accordance with one embodiment of the invention; and

FIG. 12 is a schematic plan view of an apparatus in accordance with the invention in use.

### DETAILED DESCRIPTION

In the following, various embodiments of the present invention are generally described with reference to hand surgery. It is, however, to be understood that the present invention can also be used with other types of surgical procedures that require restraining and/or irrigating a portion of an extremity of a patient's body.

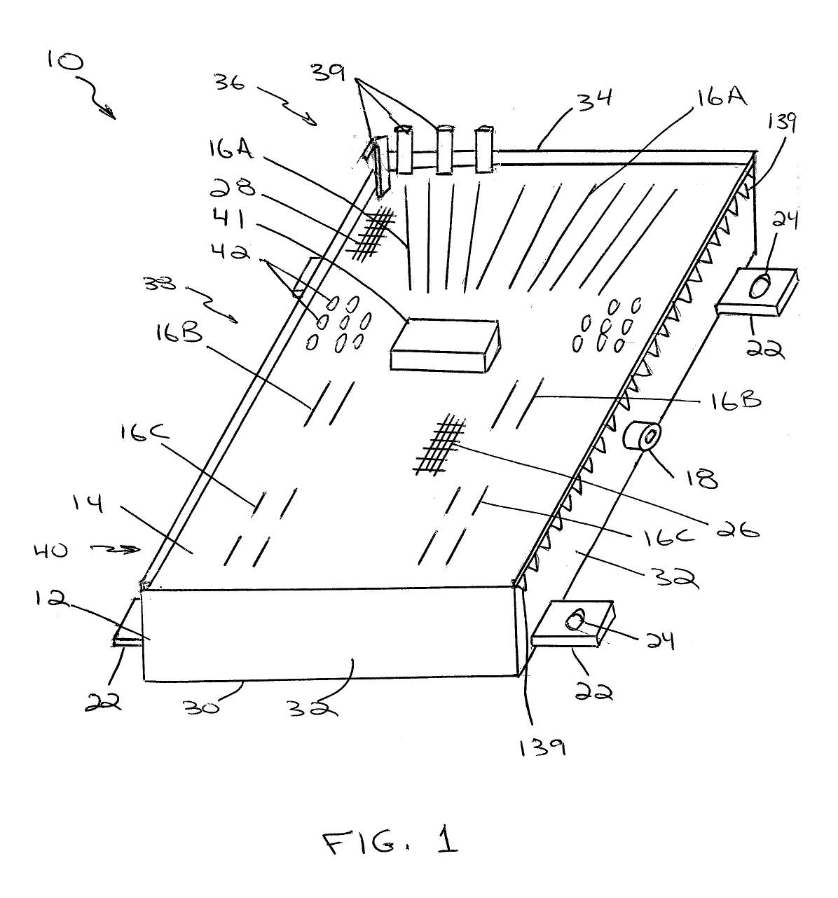

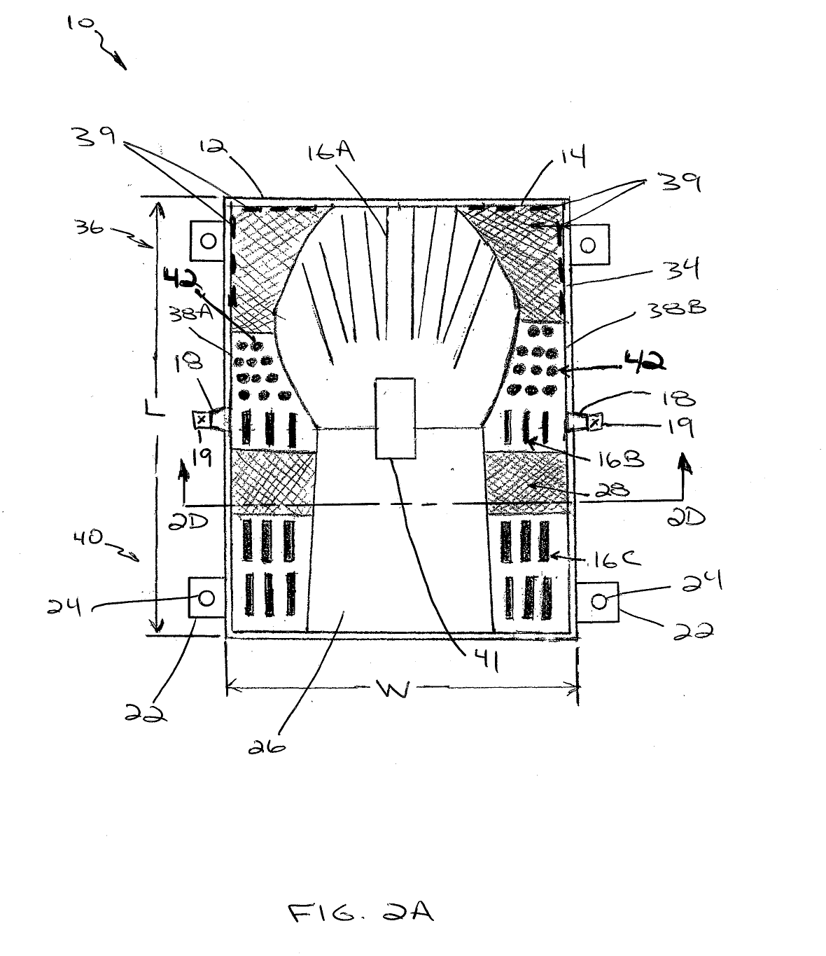

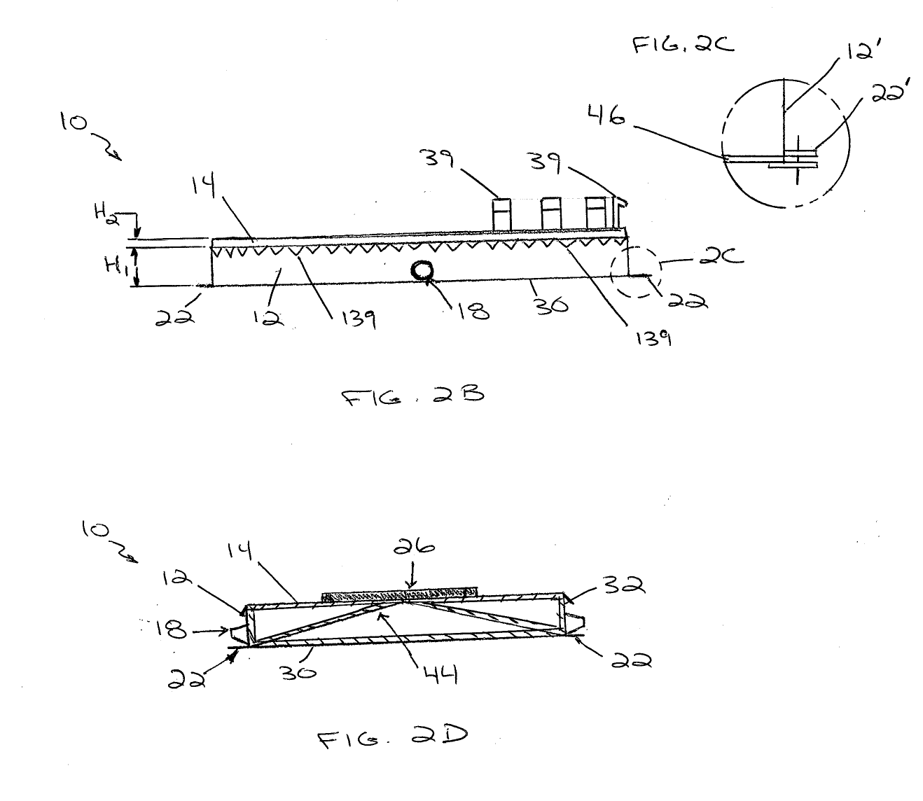

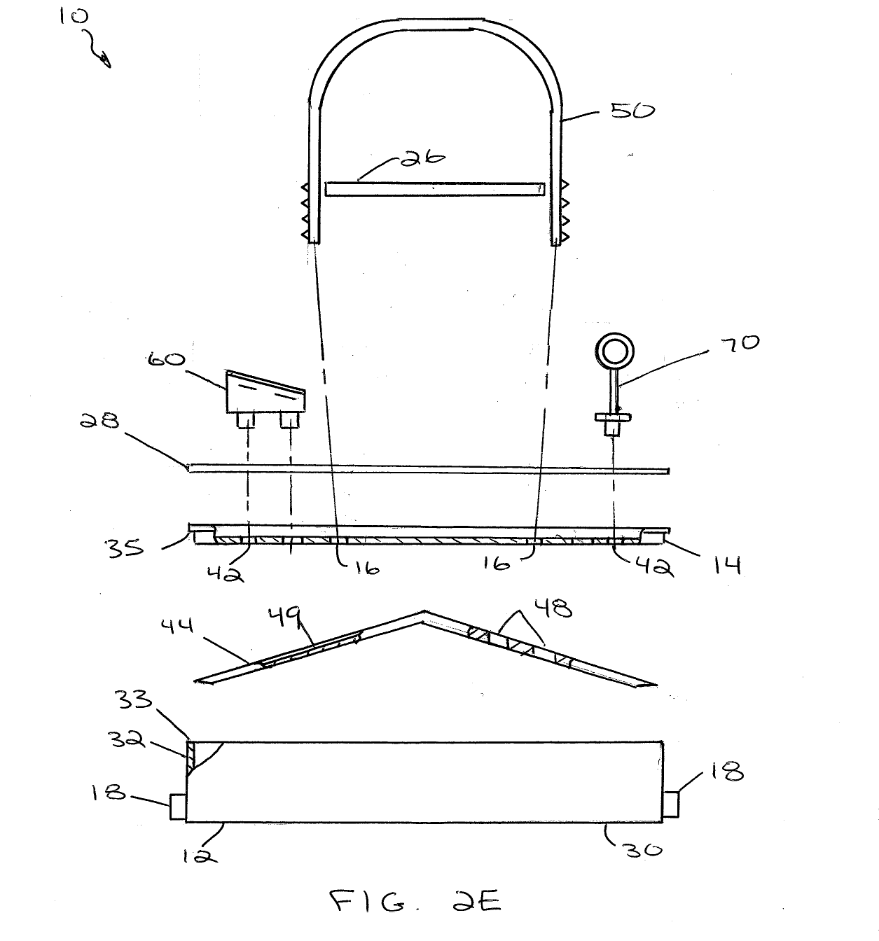

FIG. 1 is a schematic perspective view of an irrigation and retraction platform 10 in accordance with one embodiment of the invention. Generally, the platform 10 includes a chamber 12 and a support platform 14. The platform 10 shown in FIG. 1 has a substantially rectangular shape; however, the overall shape of the platform 10 can vary to suit a particular type of procedure and the anatomy of the patient. For example, the platform 10 can be circular, polygonal, or combinations thereof. The various components of the platform 10 are shown and described in greater detail with respect to FIGS. 1 and 2A-2E.

The chamber 12 is configured to capture and drain fluid and includes a bottom surface 30 and four side walls 32. The number of side walls 32 will vary depending on the shape of the platform 10 and the chamber 12. The chamber 12 includes at least one opening 18 extending through at least one of the bottom surface 30 and/or the side walls 32. As shown in the figures, the chamber 12 includes two openings 18 located at approximately the midpoint of the sides of the chamber 12. The openings 18 will be arranged for the efficient collection and transfer of fluid and other debris collected within the chamber 12. The openings can include fittings for mating with a drainage hose and/or a suction system within the operating room. The openings 18 can also include valves 19 for isolating the chamber 12 from the drainage system.

The chamber 12 can also include optional feet 22 with or without means for securing the chamber 12 to a surgical table (46 in FIG. 2C). In one embodiment, the feet 22 include a clearance hole 24 for a fastener that can be used to secure the platform 10 in place, for example, to a surgical table with corresponding receptacles for receiving the fastener. In another embodiment, the feet may be replaced by a clamp 22′ (FIG. 2C) for securing the platform 10 to the table 46. Additionally, or alternatively, the feet 22 could include an adhesive coating on a bottom surface thereof that can be used to secure the platform 10 in place.

Furthermore, the chamber 12 can include a drip tray 44 disposed therein. The drip tray 44 can be integral with the chamber 12 or an additional component disposed within the chamber 12. The drip tray 44 is shown in greater detail in FIGS. 2D and 2E. Alternatively, the drip tray 44 can be attached to a bottom surface of the support platform 14 and extend downwardly therefrom. Generally, the purpose of the drip tray 44 is to collect and direct irrigation fluid and contamination. In the embodiment shown, the drip tray 44 includes two planes forming an inverted V-shape. The drip tray 44 can be attached along all of its outer margins to the interior of the chamber 12. The drip tray 44 can include a smooth or channeled surface to help direct fluid to the openings 18 in the chamber 12. Additionally or alternatively, the drip tray 44 can include one or more openings 48 extending therethrough. The openings 48 and channels 49 can aid in the transport of fluid to the chamber 12.

In one embodiment, the chamber 12 has a length (L) of about 12 inches to about 18 inches (30-45 cm), a width (W) of about 6 inches to about 14 inches (15-35 cm), and a height (H₁) of about 1 inch to about 3 inches (2-5-7.6 cm). The chamber 12 can be manufactured of a polymer, such as polyurethane, silicones, polyethylenes, nylons, polyesters and polyester elastomers, either with or without reinforcement, a metal, such as stainless steel or aluminum, or combinations thereof. Additional materials and manufacturing methods are described in greater detail hereinbelow. In one embodiment, the chamber 12 is manufactured of a polymer material and includes an upper edge 33 made of metal and/or rubber to facilitate mating and sealing with the support platform 14 or an optional enclosure.

The support platform 14 has a substantially planar top surface and is generally divided into a distal portion 36, a medial portion 38, and a proximal portion 40. In one embodiment, the support platform 14 can gently slope from the longitudinal sides to a longitudinal midpoint of the platform 14 to assist in drainage of irrigation fluid. The support platform 14 includes a plurality of perforations that are both longitudinal (perforations 16 of varying lengths and widths) and circular (holes 42) (collectively “openings”) disposed therethrough and configured to receive various types of restraints (50, 60, 70 in FIG. 2E), as described hereinbelow. The support platform 14 can also include a gel pad 26 and a mesh layer 28.

The gel pad 26 can be a foam or silicone gel pad, such as model no. EL070, as is available from Burlington Medical Supplies, Inc. in Newport News, Va. The gel pad 26 can be used to relieve pressure on the extremity. In one embodiment, the gel pad 26 is hydrophobic. The gel pad 26 can be attached along a longitudinal axis of the support platform 14. The shape of the gel pad 26 is roughly the shape of the extremity to be supported, thus the gel pad 26 will protect the extremity from pressure during a long procedure. In addition, the gel pad 26 can include longitudinal perforations corresponding to any of the longitudinal perforations disposed beneath the gel pad 26, so as not to impede the availability of the openings for restraining and/or retracting a portion of the extremity. For example, a portion of the gel pad 26 corresponding to the area where a hand will be restrained will also have longitudinal openings so that the fingers can be immobilized separately. Alternatively, the gel pad 26 can cover substantially the entire top surface of the support platform 14 and include openings, perforations, etc. corresponding to the openings, perforations, etc. in the support platform 14. Alternatively or additionally, at least a portion of a top surface of the gel pad 26 can be sloped to promote drainage of fluid that might otherwise collect on the gel pad 26.

The mesh layer 28 can be disposed over the entire top surface of the support platform 14 or only where the there are openings therethrough. The mesh layer 28 serves to prevent any surgical instrument or device (e.g., a screw) from inadvertently passing through an opening 16, 42, while still allowing fluid to pass therethrough. The mesh layer 28 is configured to be punctured by the insertion of a restraint through the openings 16, 42. The support platform 14 can include additional openings for facilitating drainage of fluids that may collect on the surface of the support platform 14. In one embodiment, the mesh size is #4.

The longitudinal openings 16 vary in size, number, and orientation to suit a particular portion of the patient's body to be restrained. A first portion of openings 16A are arranged in the distal portion 36 of the support platform 14 and are sized and oriented to receive multiple finger restraints. As shown in FIGS. 1 and 2A, the openings 16A are arranged in five double rows Each opening 16A measures about 3 inches to about 4 inches (7.5-11 cm) in length by about ⅛ inch to about ¼ inch (3-6 mm) in width; however, the size of the openings can vary to suit a particular patient (e.g., adult or pediatric) and/or procedure. In addition, the openings 16A can be oversized in length to allow the surgeon to reposition the restraint during the procedure or use multiple restraints with each pair of openings 16A. Each double row of openings corresponds to a single finger, with about one inch (2.5 cm) spacing between the individual openings 16A. The central row is generally located on a midline of the support platform 14 and intended to restrain the middle finger, with at least two adjoining sets of rows on either side. These adjoining rows of openings 16A radiate outward from the middle openings like spokes from a wheel. This allows for either the right or the left hand to be placed on the support platform 14. Additional rows of openings 16A can be included to, for example, accommodate multiple orientations of the hand and fingers or for receiving restraints that may be used to retract a portion of tissue.

As shown in FIGS. 1 and 2A, a single row of longitudinal openings 16B are disposed in the medial portion 38 of the support platform 14. Three openings are arranged on a left medial portion 38A and three corresponding openings 16B are arranged on a right medial portion 38B. Each corresponding pair of openings 16B are sized and arranged to receive a restraint for, for example, a patient's wrist. The openings are about 1 inch to about 2 inches (2.5-5 cm) in length and about ⅛ inch to about ¼ inch (3-6 mm) in width. All of the longitudinal openings 16 can be oversized in length to allow the surgeon flexibility in locating the restraint or can be sized to provide a snug fit with the restraint in the longitudinal direction.

Also arranged in the left and right medial portions 38A, 38B distally of the openings 16B are the afore-mentioned holes 42. The holes 42 are closely spaced in the area between the gel pad 26 and an edge of the support platform 14. The holes 42 may be evenly spaced in a grid-like pattern. The holes 42 are sized and arranged to accept various restraints that can be used to, for example, restrain the patient's thumb or securely retract tissue. In one embodiment, the holes 42 are about ¼ inch (6 mm) in diameter. Using a plurality of holes 42 gives the surgeon a variety of options with respect to positioning the restraint.

Arranged in the proximal portion 40 of the support platform 14 are two rows of longitudinal openings 16C. There are two rows of three openings 16C on the left side of the proximal portion 40 and two rows of three corresponding openings 16C on the right side of the proximal portion 40 of the support platform 14. Corresponding pairs of openings 16C are sized and arranged to receive restraints for securing, for example, a patient's forearm. In one embodiment, the openings 16C are about 1¾ inches to about 2½ inches (4.4-6.2 cm) in length and about ⅛ inch to about ¼ inch (3-6 mm) in width; however, these dimensions may vary to suit restraints of different sizes for different extremities.

The support platform 14 is a substantially planar surface having a side wall 34 with a height (H₂) of about ¼ inch to about ½ inch (6-12 mm) and a width of about ¼ inch to about ½ inch (6-12 mm). The side wall 34 can extend along at least three sides of the support platform 14. In one embodiment, the side wall 34 is at least partially omitted from the proximal end of the platform 14 to provide an uninterrupted planar surface to the extremity. The length and width of the support platform is slightly less that the length and width of the chamber 12. As shown in the exploded view of FIG. 2E, the support platform 14 includes a lip 35 that engages the upper edge of the chamber 12 to support and, in some embodiments seal, the support platform 14 to the chamber 12. In this embodiment, the support platform 14 is partially nested within the chamber 12. In an alternative embodiment, the lip 35 can be disposed on a lower edge of the support platform 14, such that the support platform 14 does not significantly protrude into the chamber 12. Alternatively, the support platform 14 can be permanently affixed to the chamber 12.

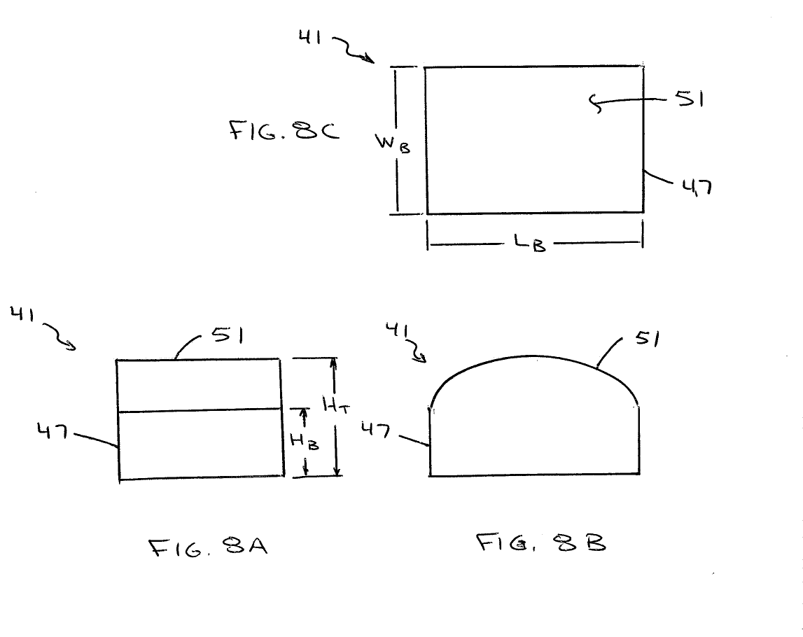

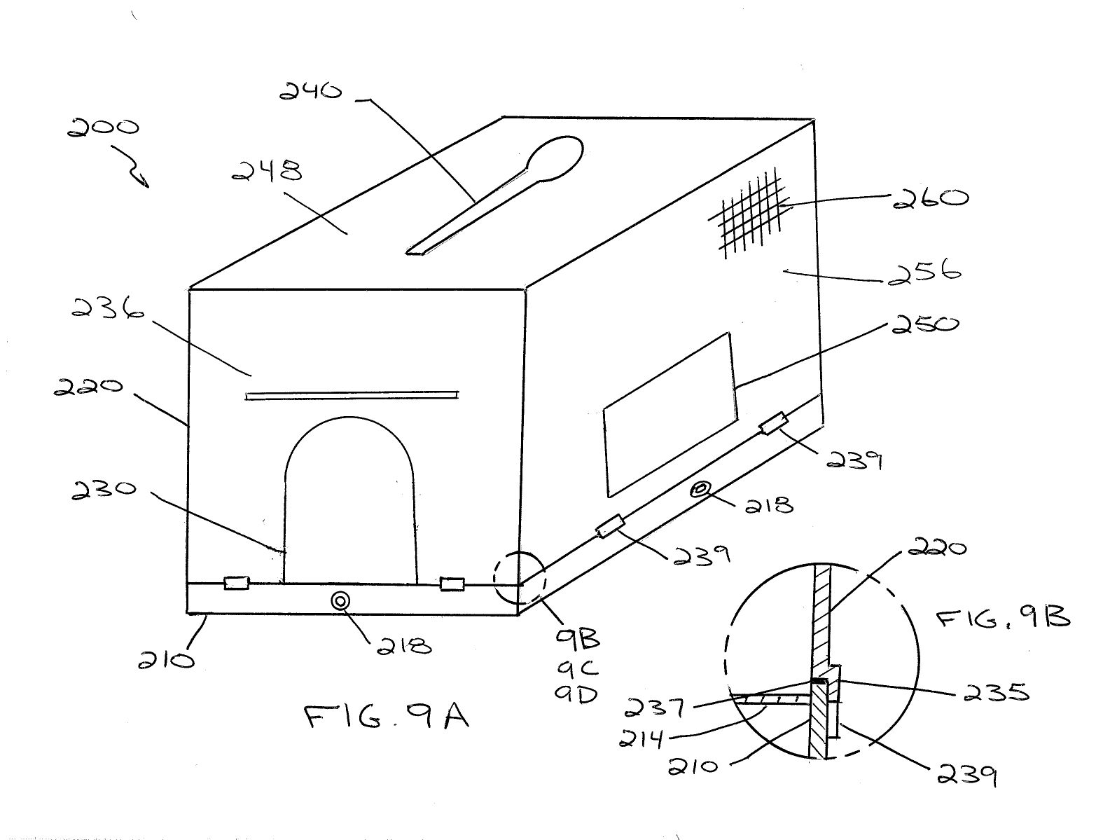

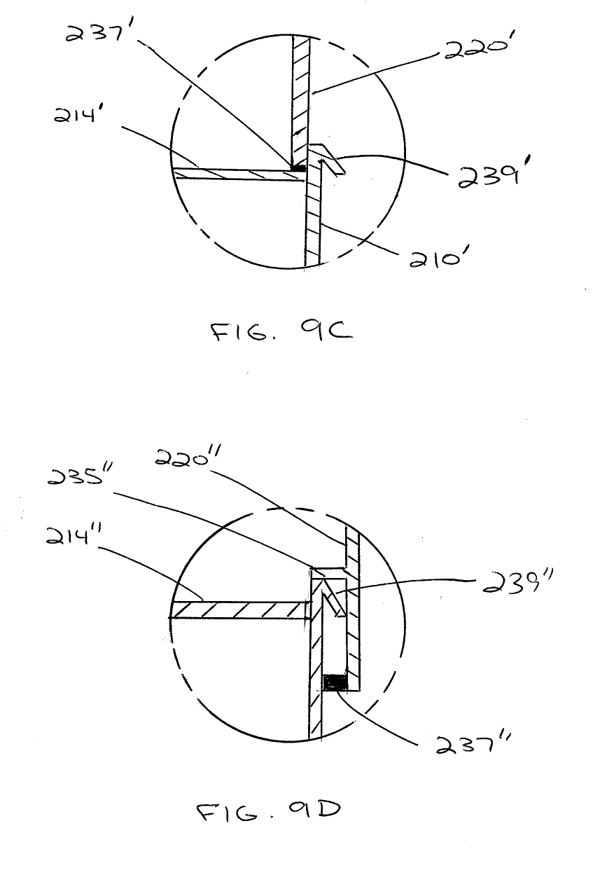

Additional optional features include suture retraction pegs 39, 139 and a positioning pad 41. As shown in FIGS. 1, 2A, and 2B, a plurality of suture retraction pegs 39, 139 are disposed along the periphery of the support platform 14. The pegs 39, 139 can be disposed along the entire periphery of the platform 14 or only a portion thereof. Alternatively or additionally, the pegs 39, 139 can be disposed along all or a portion of the periphery of the chamber 12. For example, the pegs 39 can extend upwardly from the walls 32 of the chamber 12. In another embodiment, the pegs 139 extend down and away from the periphery of the chamber 12. The pegs 39, 139 are described in greater detail with respect to FIGS. 7A-7C. The positioning pad 41 is described in greater detail with respect to FIGS. 8A-8C. The pad 41 can be positioned at essentially any location on the support platform 14, as necessary to accommodate the particular anatomy of the patient. The pad 41 can be secured in place on the support platform 14 by, for example, an adhesive, friction, hook and loop type fastener, or other mechanical means.

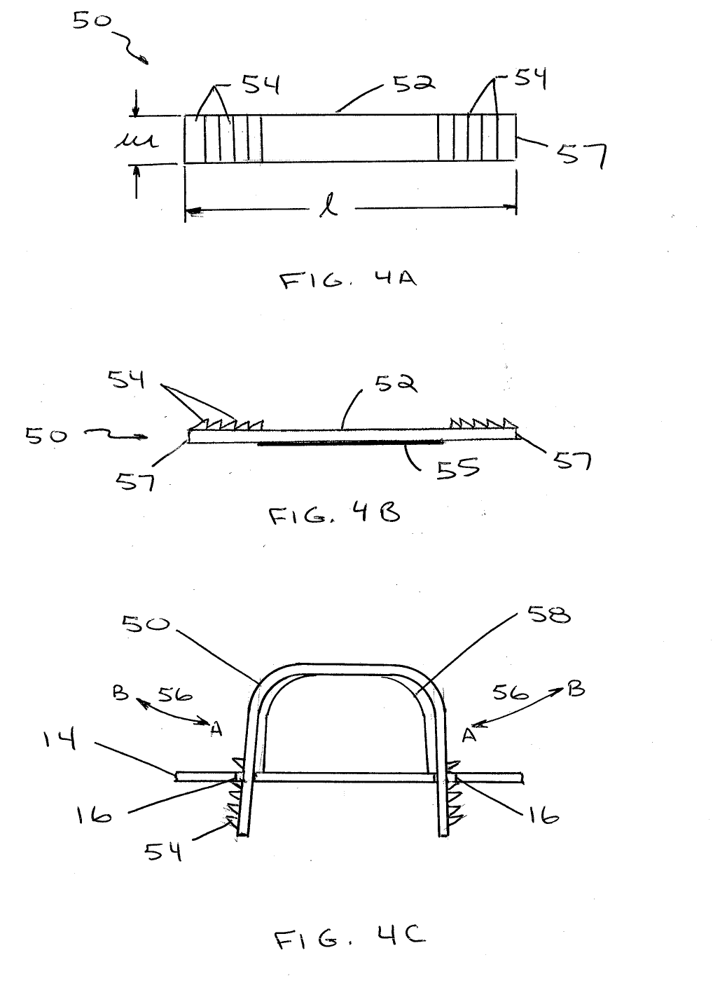

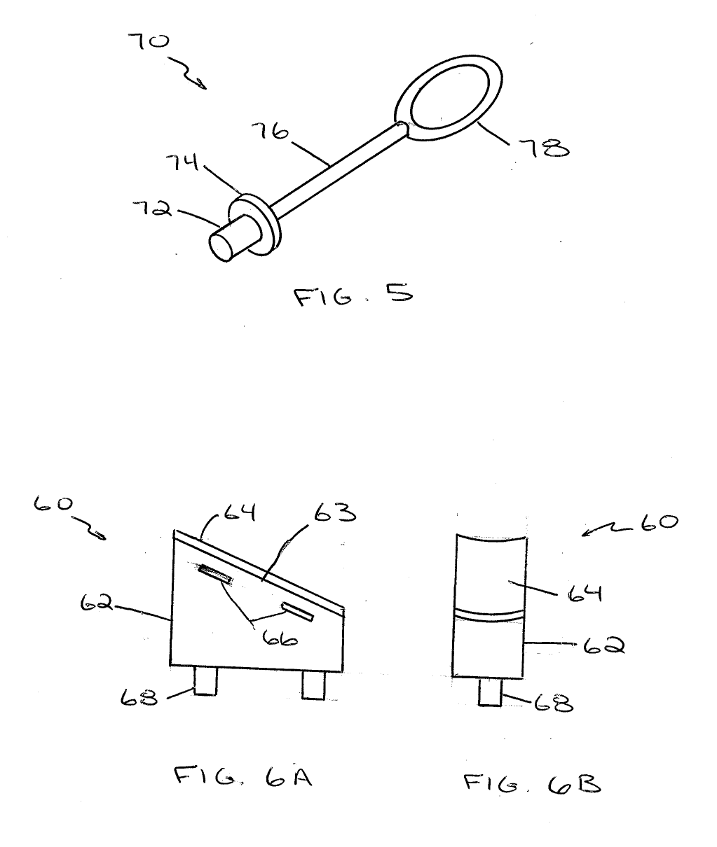

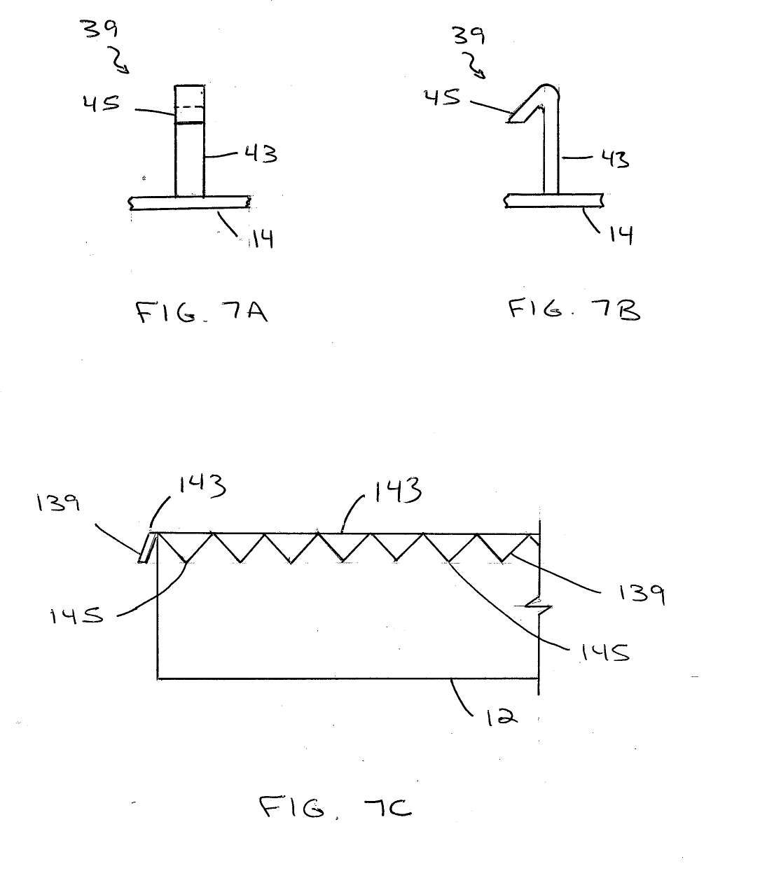

FIG. 2E depicts an exploded view of the platform 10, with a variety of restraints depicted. As shown, the platform 10 includes an optional support block 60 (FIGS. 6A and 6B), a thumb restraint 70 (FIG. 5) and a forearm restraint 50 (FIGS. 4A-4C). The various restraints/block include projections, as will be discussed in greater detail hereinbelow, that mate with the openings 16, 42 in the support platform 14. With respect to the support block 60 and thumb restraint 70, their projections may be sized to provide a friction or force fit with their corresponding opening 42. Additionally or alternatively, the projections can be secured to the support platform 14 by mechanical means (e.g., a fastener) or an adhesive. Besides the patient's extremity, the various restraints can also be used to secure other tissue (e.g., retract flaps of skin).

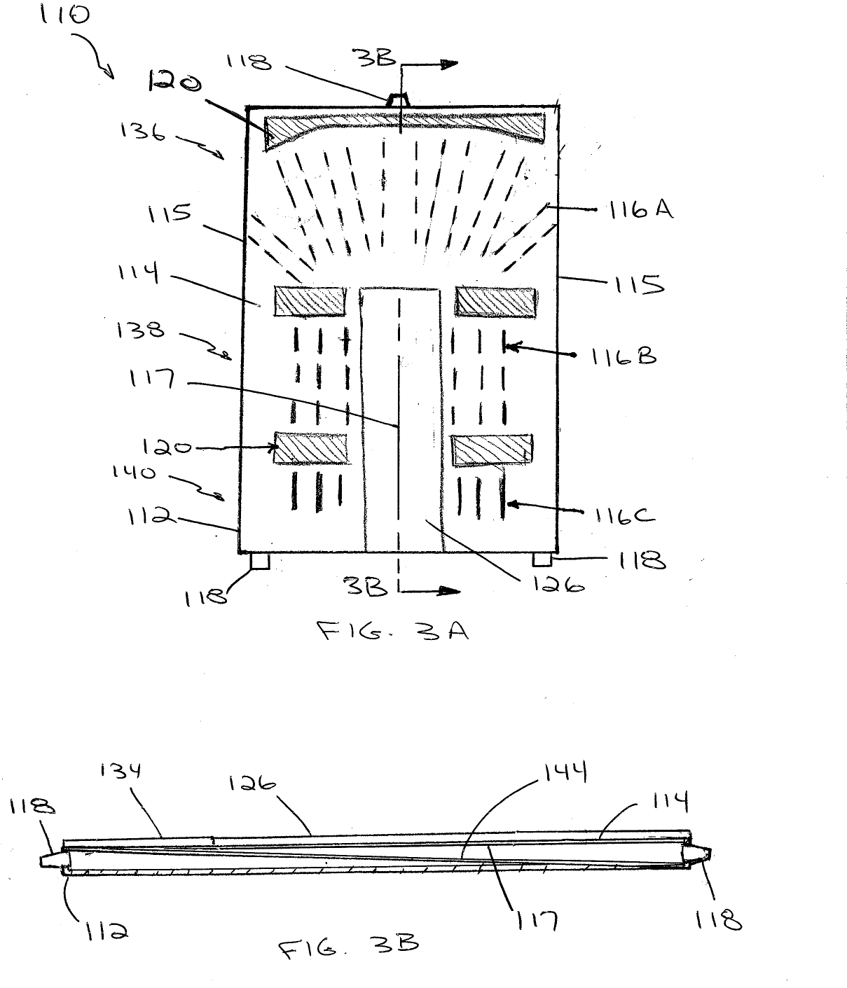

FIGS. 3A and 3B depict an alternative platform 110. The platform 110 is similar to the platform 10 described hereinabove, but includes a different arrangement of components. The platform 110 includes a chamber 112 with a substantially flat drip tray 144 that is inclined lengthwise to a proximal end of the chamber 112, where two drain openings 118 are located. The drains 118 can be hooked to wall suction to collect irrigation fluid and can include fittings and/or valves as previously described. The drip tray 144 can be attached to the sides of the chamber 112. One or more additional drain openings 118 can be included at the distal end of the chamber 112 to further assist in draining irrigation fluid from the chamber 112.

The platform 110 also includes a support platform 114. The support platform 114 slopes gradually from its longitudinal sides 115 to the longitudinal midpoint 117 of the platform 114. The slope is gradual enough so as not to affect use of the support platform 114, but sufficient to assist in drainage of irrigation fluid. The distal portion 136 of the support platform 114 includes multiple longitudinal openings 116A that measure about ½ inch (12 mm) in length and about ⅛ inch (3 mm) in width. Each opening is positioned about ½ inch to about 1 inch (1.2-2.5 cm) apart (both side by side and top to bottom). These multiple openings 116A are sized to receive restraints for holding the patient's fingers. The openings 116A are arranged in a radial pattern to provide the surgeon with multiple configurations for restraining the patient's fingers. The unused openings 116A will allow passage of irrigation fluid to the drip tray 144 and chamber 112. In addition, there are larger drainage openings 120 present. These will be positioned at the distal and medial portions of the support platform 114. The openings 120 may also include mesh coverings.

The medial portion 138 of the support platform 114 includes three rows of pairs of openings 116B that are about 1 inch (2.5 cm) in length and about ⅛ inch (3 mm) in width. These openings 116B are positioned about 1 inch (2.5 cm) apart both side by side and top to bottom. The patient's wrist and distal forearm will be positioned over this area and held by restraints inserted into these openings 116B.

In the proximal portion 140 of the support platform 114 are larger openings 116C that are about 2 inches (5 cm) in length and about ¼ inch (6 mm) in width. These openings 116C are sized and arranged to receive restraints for immobilization of the patient's proximal forearm.

The platform 110 can also include a gel pad 126 that measures about 4 inches (11 cm) in width, about ½ inch (12 mm) in thickness, and about 12 inches (30 cm) in length and overlies a central portion of the platform 114 to support the patient's forearm and hand. Alternatively, the gel pad 126 can extend over all or any portion of the support platform 114.

One type of restraint 50 that can be used for securing the patient's arm, wrist, finger, or other extremity is depicted in FIGS. 4A-4C. The restraint 50 includes an elongate body or strap 52 and a series of projections 54 arranged on each end 57 of the strap 52. The restraint 50 can be made of a relatively soft, malleable plastic and can include additional padding 55 to further provide comfort to the area of the patient's body to be restrained. The padding 55, which could be in the form of a gel pad, would be disposed on the side of the strap 52 opposite the side with the projections 54, as this side will be in contact with the patient's body. It is desirable for the material used for the restraint 50 to be radiolucent so as not to interfere with or require removal for imaging (e.g., the taking of x-rays) during the procedure.

The projections 54 can be generally thickened areas that run transversely to the elongate strap 52 and are about ⅛ inch (3 mm) in thickness. In the embodiment shown, each thickened area is substantially in the shape of a right angle triangle; however, other shapes are contemplated and within the scope of the invention, such as, for example, arcuate projections.

The restraints 50 can have varying lengths (l) and widths (w) to suit a particular application and patient size. For example, restraints 50 for immobilizing the patient's proximal forearm can be about 1½ inches to about 2 inches (3.75 to 5 cm) in width and about 8 inches to about 14 inches (20-36 cm) in length. For the distal forearm, including the wrist, each restraint 50 can be about ¾ inch to about 1¼ inch (2-3 cm) in width by about 6 to about 10 inches (15-25 cm) in length. For immobilizing the patient's fingers, the restraints can be about 3 inches to about 4 inches (7.6-10 cm) in length and about ¼ inch to about ¾ inch (6-18 mm) in width.

In use, the ends 57 of the restraints 50 are flexed toward each other (see arrows 56A in FIG. 4C) with the projections 54 on the outer side until the ends 57 substantially align with the corresponding openings 16. The ends 57 are then inserted into the openings 16 in the support platform 14. Because the strap 52 is biased into a planar orientation, the ends of the restraint 50 will attempt to regain their original orientation (see arrows 56B in FIG. 4C), thereby causing the projections 54 to engage the outer edges of the openings 16.

The fit of the restraints 50 can be adjusted by varying the length of insertion. Because there are multiple projections 54, there are multiple insertion positions, thereby immobilizing the part 58 (forearm, wrist, fingers) under correct tension. For example, the restraint 50 can be tightened by further inserting the ends 57 of the restraint 50 through the openings 16 and engaging a projection 54 positioned further away from the end 57 of the strap 52. Conversely, the restraint 50 can be loosened, or even removed, by partially retracting or removing the ends 57 from the openings 16. The restraint 50 can be removed by pinching the curved strap to bring the ends 57 of the restraint 50 closer together (see arrows 56B in FIG. 4C), thereby disengaging the projections form the outer edges of the openings 16. Once the projections 54 are disengaged, the ends 57 of the restraint 50 can be slid out of the openings 16, either completely or to a new position (i.e., engagement point with a projection 54). In addition, because the longitudinal openings 16 are longer than the widths of the various straps for which the openings 16 are sized, the restraints 50 can be repositioned along a length of the part 58 being immobilized, for example, during a procedure to expose different areas of the part 58 to the surgeon.

The thumb can be retracted in two ways. The simplest way, typically used when the thumb is not being directly operated on, is by use of a thumb restraint 70, as depicted in FIG. 5. The restraint 70 includes a flange 74, a projection 72 extending from one side of the flange 74 and an elongate body or strap 76 extending from an opposite side of the flange 74. At the end of the strap 76 is a loop 78 for receiving the thumb. In one embodiment, the loop 78 is in the form of a circle measuring about 3 inches (7.6 cm) in circumference or about 1 inch (2.5 cm) in diameter and about ⅛ inch (3 mm) in thickness. The flange 74 is about ½ inch (12 mm) in diameter and about ⅛ inch (3 mm) in thickness. The strap is about 1 inch (2.5 cm) in length. The projection 72 is about ¼ inch (6 mm) in length and about ¼ inch (6 mm) in diameter. The projection 72 is sized to fit into one of the holes 42 in the support platform 14, as depicted in FIG. 2E. The size and shape of the thumb restraint 70 and its various components will vary to suit a particular patient (e.g., adult or pediatric).

An alternative means of immobilizing the thumb when surgery is performed in this area is depicted in FIGS. 6A and 6B. FIG. 6A is a side view and FIG. 6B is an end view of a support block 60 for use with a support platform 14 in accordance with one embodiment of the invention. The support block 60 has a generally triangular shaped body 62 and openings 66 for receiving straps or other types of restraining devices. A small portion of the apex of the triangle has been removed so that the block 60 has a 4-sided body 62. The openings 66 can be through holes through which a conventional strap can be passed. The opening 66 can be configured to receive a strap about ¼ inch to about ½ inch (6-12 mm) in width and about 3 inches to about 4 inches (7.6-11 cm) in length. The strap can be secured by, for example, a hook and loop type fastener, such as the Velcro® brand sold by Velcro Industries B.V. Alternatively, the openings 66 can be configured to receive a restraint 50 as previously described.

The block 60 further includes an upper inclined surface 63 curved to cradle the thumb. The upper curved surface 63 of the block 60 can also include a gel pad as previously described. On the bottom surface of the thumb block 60 are two projections 68 that measure about ¼ inch (6 mm) in length and diameter. As discussed hereinabove with respect to the thumb restraint 70, the projections 68 are configured to engage any two adjacent the holes 42 in the support platform 14. Two projections 68 are used to prevent the thumb block from rotating or moving. In one embodiment, the block 60 is about 2 inches (5 cm) in length, about 2 inches (5 cm) in height on the short side, about 2¼ inches (5.7 cm) in height on the inclined side, and about ¾ inches (2 cm) in thickness.

FIGS. 7A and 7B depict one embodiment of the optional suture retraction peg 39. Generally, the peg 39 includes a base 43 and a hook end 45 extending outwardly from the top of the base 43. The peg 39 is used to attach, for example, a wound retraction suture. A loop formed in the suture is captured and retained by the hook end 45. A plurality of pegs 39 can be spaced along the periphery of the platform 14 and/or the chamber 12, for example about every ½ inch (12 mm). In one embodiment, the peg 39 is about ¼ inch (6 mm) in width, about ⅛ inch (3 mm) in thickness, and about ½ inch to about ¾ inch (12-18 mm) in height. The hook end 45 can extend from the base 43 at an angle from about 30 degrees to about 60 degrees and for a distance of about ¼ inch (6 mm). As shown, the base 43 extends substantially vertically upward from the support platform 14; however, the base 43 could extend upwardly at an angle to the platform 14 and/or the chamber 12. The specific size, shape, and configuration of the peg 39 may vary to suit a particular application. The peg 39 can be formed integrally with the support platform 14 or chamber 12 or could be mechanically attached thereto. In addition, the peg 39 could be attached to the support platform 14 in the same manner as any of the restraints described herein.

An alternative suture retraction peg 139 is depicted in FIG. 7C. The pegs 139 are generally flat and triangular in shape and extend down and away from the periphery of the chamber 12. Alternatively, the pegs 139 could extend from the periphery or sidewall of the support platform 14. In use, retracting sutures can be wrapped around the peg 139 and tied in place or secured between pegs 139 by a friction fit.

The pegs 139 include a base 143 that is attached to the periphery of the chamber 12. The pegs 139 can be fused or otherwise bonded to the periphery of the chamber 12 or can be formed as part of the chamber 12 by, for example, injection molding. The pegs 139 include a pointed end 145 that in one embodiment projects about ¼ inch to about ½ inch (6-12 mm) from the periphery and are spaced about ½ inch to about 1 inch (1.2-2.5 cm) apart. The pegs 139 can extend from the chamber 12 at an angle of about 30 degrees to about 60 degrees from the sidewall of the chamber 12. The pegs 139 can extend along at least three sides of the platform 10. Typically, pegs 139 would not be located on the proximal end of the platform 10 where the extremity rests. In one embodiment, the triangular shape of the peg 139 has a base dimension of about ¼ inch to about ½ inch (6-12 mm) and a height of about ½ inch to about 1 inch (1.2-2.5 cm). The specific size, shape, and configuration of the peg 139 may vary to suit a particular application.

The optional positioning pad 41 can be used to support and position the patient's extremity during the operation. For example, the pad 41 can be used provide flexion to the wrist when operating on the dorsum of the hand/wrist. Additionally, the pad 41 can be used to provide support for the hand as necessary when operating on the dorsum of the hand/fingers.

The pad 41 shown in FIGS. 8A-8C has a polygonal shape with a base 47 and a curved top surface 51. The shape of the pad 41 can include any combination of polygonal and arcuate elements. For example, the base 47 could have a generally rectangular shape tapered at one end with a slanted top surface 51. Alternatively, the top surface 51 could be a planar horizontal surface. In one embodiment, the base 47 of the pad 41 has a length (L_{b}) from about 3 inches to about 6 inches (7.5-15 cm), a width (W_{b}) from about 3 inches to about 5 inches (7.5-12.5 cm), and a height (H_{b}) from about 1 inch to about 3 inches (2.5-7.5 cm). The overall height (Hₜ) of the pad 41, including the top surface 51, can be from about 2 inches to about 4 inches (5-10 cm). The pad 41 can be made from a relatively soft, semi-spongy material that can retain its shape and provide comfort and support to the extremity. The pad 41 can include an outer layer of a firmer material and/or include a coating, for example an anti-bacterial coating.

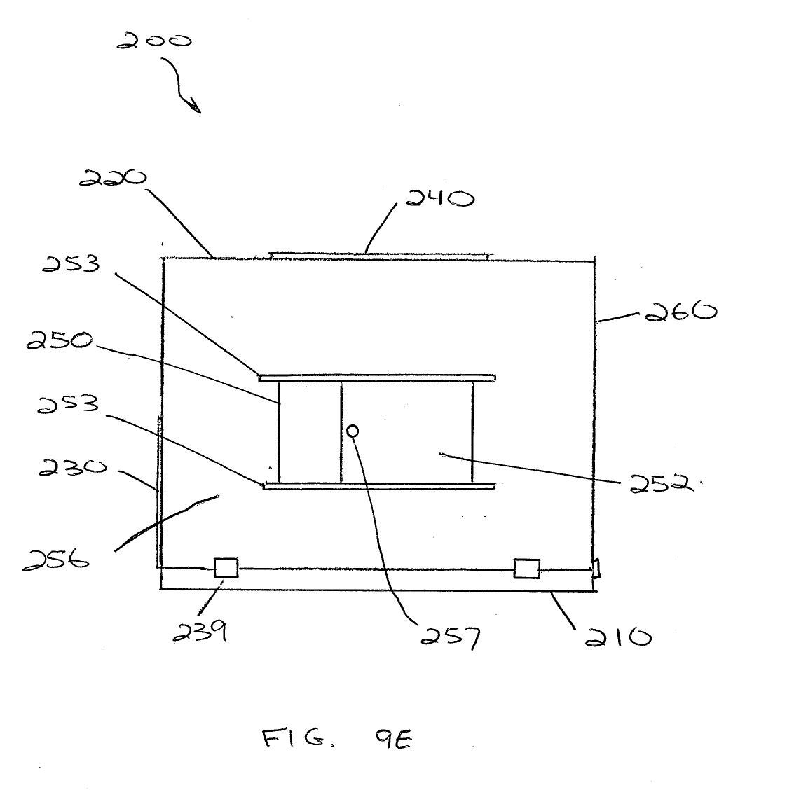

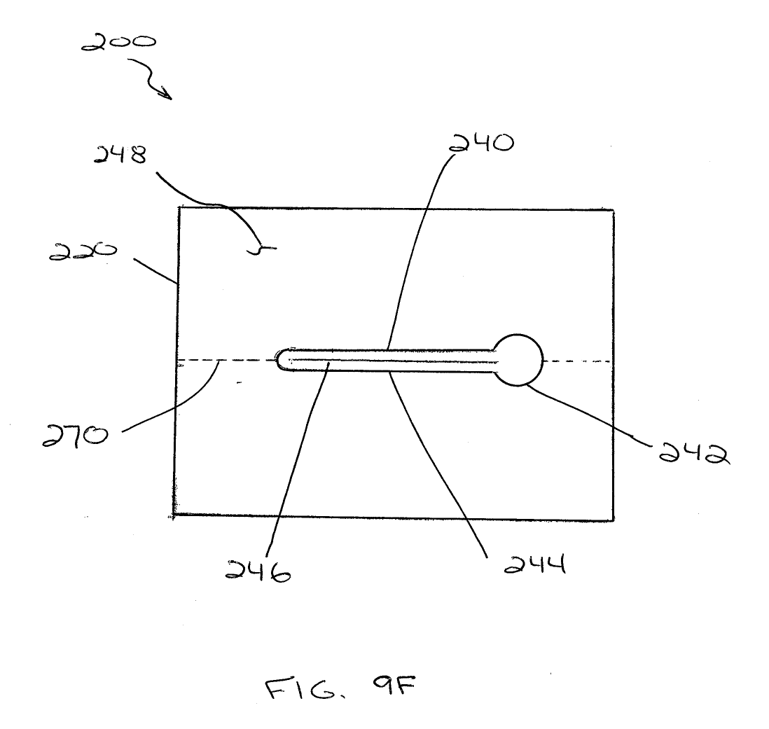

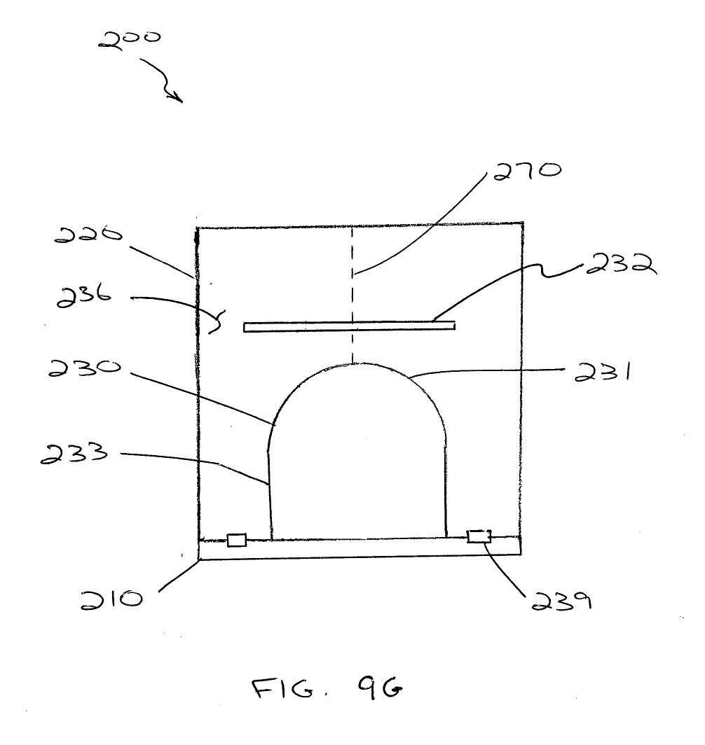

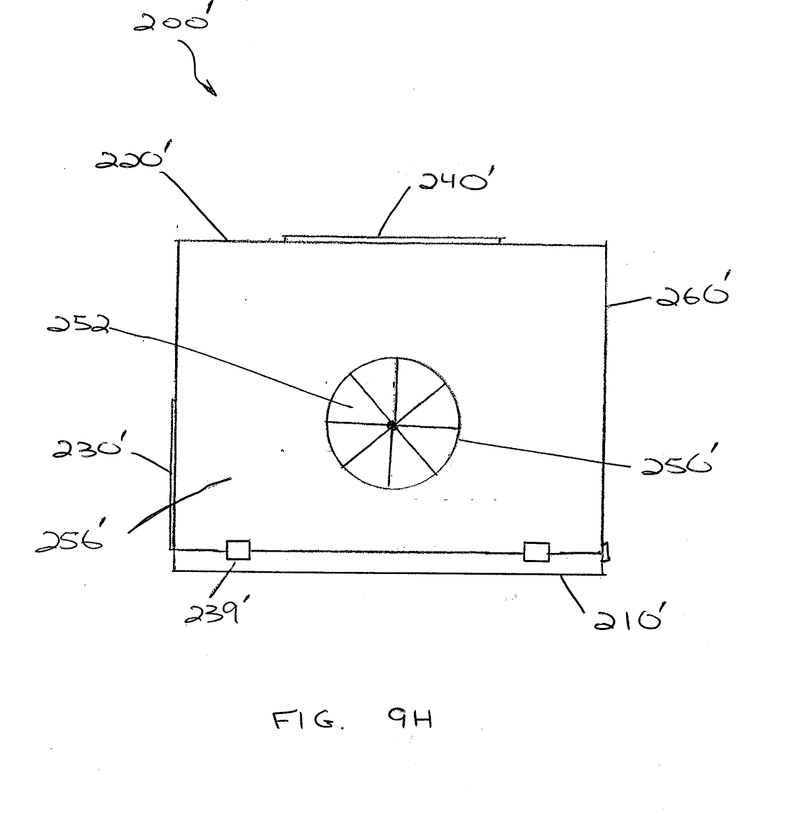

An enclosed apparatus 200 in accordance with the invention is depicted in FIGS. 9A-9H. As shown in FIG. 9A, the apparatus 200 includes a platform 210 and an enclosure 220 that engages the platform 210. The platform 210 is similar to the platforms 10, 110 described hereinabove. Generally, the bottom of the enclosure 220 fits on or over the platform 210. The exact arrangement of the enclosure 220 and its fit to the platform 210 will depend, in part, on the specific arrangement of components on the platform, for example the inclusion of suture retraction pegs. The enclosure 220 can include a notch or other cut-outs as necessary on its bottom edge to accommodate, for example, drainage ports 218 on the platform 210.

The enclosure 220 includes at least one opening 250 on the side of the enclosure 220, and typically an opening 250 on each side of the enclosure. The openings 250 are configured to provide access to the surgeon or other medical personal to the area inside the enclosure 220. In addition, a surgical device can be inserted through the openings 250. The enclosure 220 also includes an opening 230 on an end wall of the enclosure 220. The opening 230 is configured to receive the extremity of the mammalian body, for example, a patient's hand and forearm. The enclosure 220 also includes an opening 240 disposed on the top surface of the enclosure 220. This opening 240 is configured to provide access for a surgical device, such as, for example, a jet lavage wand. Furthermore, all or at least a portion of the walls 260 of the enclosure can be transparent to provide unimpaired line of sight for the surgeon, thereby ensuring effective and complete irrigation without inadvertently touching the wound.

In one embodiment, the enclosure 220 can include a lip 235 for securely engaging the platform 210, as shown in FIG. 9B. A gasket 237 can be included to provide a watertight seal between the enclosure and the platform 210. The gasket 237 can be disposed on the upper edge of a side wall of the platform 210 (e.g., the upper edge of the chamber or support platform) or can be disposed on the lip 235 of the enclosure 220. In an alternative arrangement, the sidewalls of the platform 210 and enclosure 220 can be aligned and include tongue and groove type joints, with or without a gasket disposed therebetween. In addition, the apparatus 200 can include a series of clamps 239 (or other types of mechanical fastening means) disposed about the perimeter of the platform 210 and/or enclosure 220 to removably secure the enclosure 220 to the platform 210. Alternatively or additionally, the enclosure 220 can engage the platform 210 with a press fit.

FIGS. 9C and 9D depict alternative mating arrangements of the enclosure 220 and platform 210. As shown in FIG. 9C, the platform 210′ includes suture retraction pegs 239′ located on the external surface thereof. The enclosure 220′ is configured to rest on the support platform 214′ just inside of the sidewalls of the chamber 210′. The enclosure 220′ can include a gasket 237′ located along the bottom of the enclosure sidewall to aid in sealing the enclosure 220′ and chamber 210′. The enclosure 220′ can also include fasteners as described above.

As shown in FIG. 9D, the enclosure 220″ is configured to also enclose the externally located suture retraction pegs 239″. The enclosure 220″ includes an internal lip 235″ that is configured to rest on the top of the sidewall of the chamber 210″ and/or the support platform 214″. The lip 235″ can extend around the entire perimeter of the enclosure 220″ or be located at only certain areas as needed to support the enclosure 220″ on the chamber 210″. The enclosure 220″ includes a gasket 237″ disposed between the edge of the enclosure 220″ and the side of the chamber 210″ to seal the enclosure 220″ to the chamber 210″. Additional gaskets can be included as necessary to fully seal the enclosure 220″ to the chamber 210″. For example, an additional gasket may be included at the proximal end of the chamber 210″ to compensate for the omitted sidewall portion.

In operation, the enclosure 220 and platform 210 provide a complete drainage and collection system that protects the operating team from splatter or contamination with a universal port (opening 240) for the irrigator and ports (openings 250) for the assistant's hand(s). It provides an efficient and closed system for the collection of the irrigating fluids within the enclosure 220 with no pooling or fluid collection around the wound. In addition, drainage is provided through one or more openings 218 in the platform 210 through a connection to a standard wall suction port. The openings 230, 240, 250 allow the surgeon and assistant maximum ease in accessing, exposing and treating the affected area. The enclosure 220 further eliminates the need to cover the wound with towels and the resulting lint contamination.

In one embodiment, the apparatus is about 12 inches to about 18 inches (30-45 cm) in length and about 6 inches to about 14 inches (15-35 cm) in width and about 12 inches to about 18 inches (30-45 cm) in height; however, the size and shape of the apparatus 200 will vary to suit a particular application. Once the extremity is placed on the platform 210, the enclosure 220 snaps over or is otherwise secured to the platform 210. This arrangement completely surrounds the extremity and also allows the surgeon or other operator to manipulate and retract tissues and visually inspect the area to be irrigated to reach the area effectively, rather than irrigating blindly under cover with towels.

As shown in FIG. 9E, the opening 250 in the side walls 256 is a fenestrated type opening including a cover 252 that slidably engages the side wall 256 via two horizontal tracks 253. The sliding cover 252 can include a knob 257 or similar structure to allow the user to slide the cover 252 horizontally to expose/cover the opening 250, as necessary. In one embodiment, the opening 250 has a generally rectangular shape and is about 6 inches (15 cm) in height and about 6 inches (15 cm) to about 10 inches (25 cm) in length; however, the size and shape of the opening 250 can vary to suit a particular application.

FIG. 9H depicts an alternative embodiment of the opening 250′. The opening 250′ includes a flexible cover 252′ that engages and at least partially seals around the assistant's hand and/or forearm. The cover 252′ may be a relatively flexible material, such as a thin rubber sheet, with or without pleats, that will substantially conform to the shape of the assistant's hand and/or forearm or a surgical instrument. In one embodiment, the opening is about 6 inches (15 cm) in diameter. Alternatively, the opening 250′ could be a glove port that isolates the assistant's hand from the interior of the enclosure 220′ and does not require a separate cover.

FIG. 9F depicts the top surface 248 of the enclosure 220. The opening 240 disposed in the top surface 248 includes a universal port 242 for receiving any type of irrigator, in particular pulsed irrigators. In one embodiment, the port 242 is about 4 inches (11 cm) in diameter. The opening 240 also includes a slotted transparent portion 244 for positioning the irrigator relative to the wound. For example, a jet lavage apparatus, which is approximately 12 inches (30 cm) in length, can be moved along the slotted portion 244. The slot 244 can extend substantially the entire length of the top surface 248. The opening 240 can also include a cover or gasket 246 that conforms to the shape of the irrigator or other instrument to prevent fluid from exiting the enclosure 220. The top surface 248 and gasket 246 can also be transparent so that the operative (injured) area can be visualized.

As shown in FIG. 9G, the opening 230 is slotted to the bottom edge of the enclosure 220, such that the enclosure 220 can be placed over an extremity already restrained on the platform 210. In such an arrangement, after the procedure is completed, the fasteners 239 can be disengaged and the enclosure 220 lifted off of the platform 210. The opening 230 shown is a generally inverted U-shaped aperture that includes an arcuate top portion 231 and two side walls 233 that extend downwardly therefrom. In one embodiment, the opening 230 is about 6 inches (15 cm) wide and about 8 inches (20 cm) in height. The size and shape of the opening 230 can also vary to suit a particular application. As shown in FIG. 9G, the end wall 236 includes a sterile drape 232 mounted thereon. In one embodiment, the sterile drape 232 is a towel type drape attachable to the end wall 236 by an adhesive, such as the Steri-Drape™ 1000 available from the 3M company. The drape 232 can be wrapped around the portion of the extremity exiting the opening 230.

In an alternative embodiment, the opening 230 disposed on the end wall 236 of the enclosure 220 can be a fenestrated opening including a cover (see, for example, FIG. 9E) or have a flexible cover configured to seal around the extremity placed therethrough (see, for example, FIG. 9H). In an embodiment where the extremity is placed through the opening 230, the enclosure 220 can be removed by simply cutting the enclosure 220 along a provided score line 270 between the two halves of the enclosure 220, thereby providing easy removal of the enclosure 220.

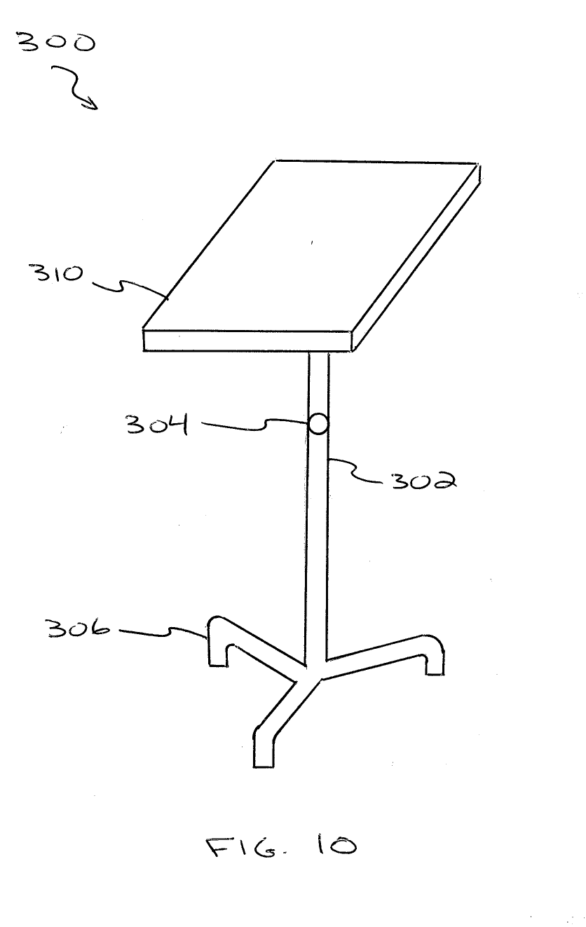

In an alternative embodiment depicted in FIG. 10, an apparatus 300 in accordance with any of the embodiments of the invention can include a pedestal 302 extending from a bottom surface of an irrigation and retraction platform 310. Such an arrangement of the apparatus 300 eliminates the need for a surgical table to support the platform 310. The platform 310 can be in accordance with any of the various platforms and apparatus described herein.

The pedestal 302 shown includes three legs/feet 306 for supporting the apparatus, however, any number and arrangement of legs/feet are contemplated and within the scope of the invention as necessary to support the platform 310. In addition, the pedestal 302 could include three of more legs directly attached to the bottom surface of the platform 310. Examples of pedestals are depicted in U.S. Pat. Nos. 3,540,719 and 4,635,914, the entire disclosures of which are hereby incorporated by reference herein. Moreover, the pedestal 302 can include means 304, such as, for example, a sliding pole and clamp system, that facilitates adjusting the height of the apparatus 300.

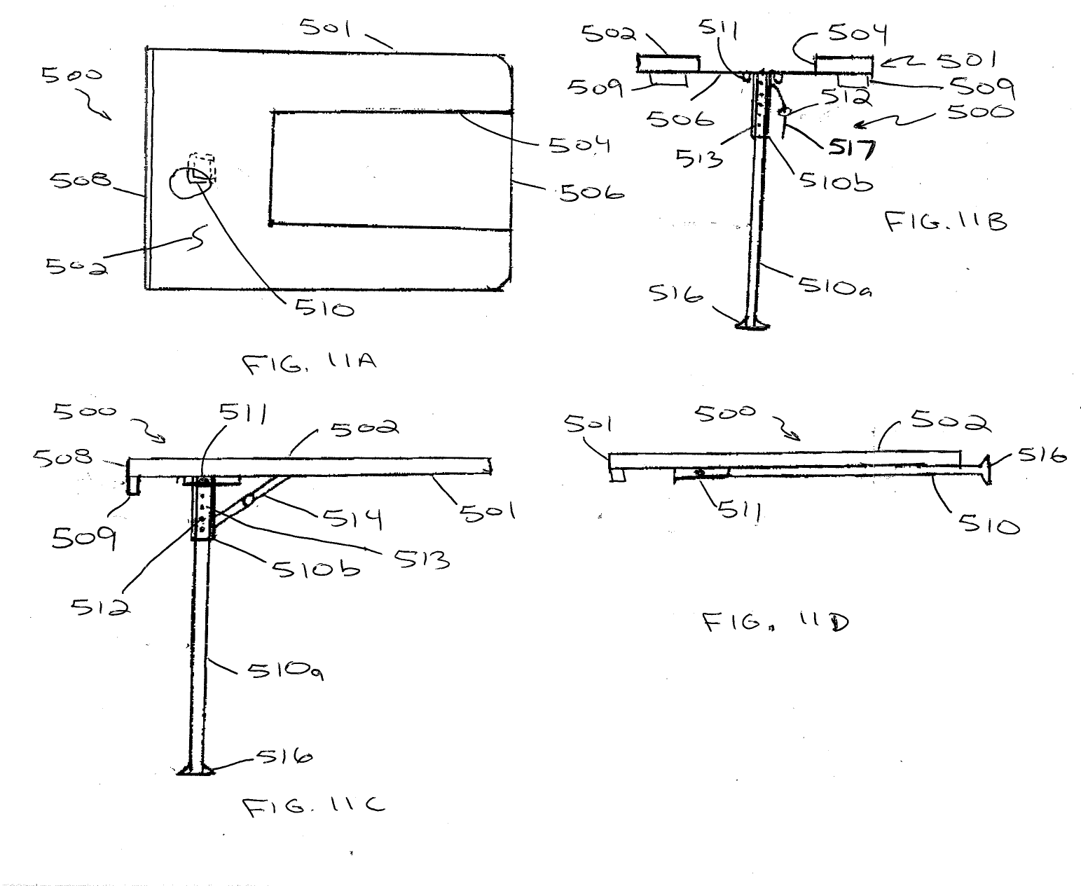

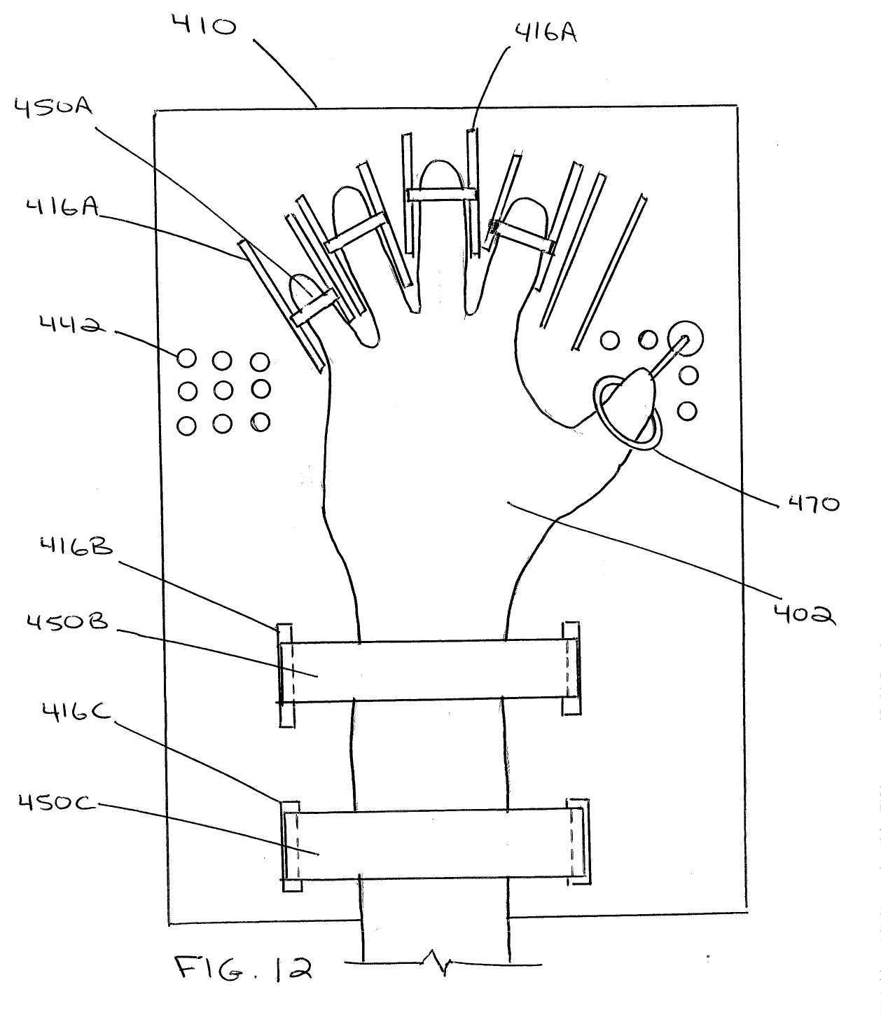

A surgical table 500, such as a hand table or arm board, designed for use with an irrigation platform as described herein is depicted in FIGS. 11A-11D. The table 500 can be supported solely by attachment to an operating room (OR) table, as is known from conventional surgery tables, or include one or more support legs. In one embodiment, the table 500 is about 24 inches (60 cm) in width, about 32 inches to 36 inches (80-90 cm) in length, and about 2 inches (5 cm) in thickness. The table 500 can include chamfered or rounded corners to reduce or eliminate the possibility of injury to personnel working proximate the table 500. The materials used for the table 500 and its various components can be radio-lucent to enable the use of X-ray or other imaging equipment.

Generally, the table 500 is configured for attachment to the OR table by, for example, two rectangular bars 509 attached to an inferior surface of a proximal end 508 of the table 500. In one embodiment, the bars 509 are about 2 inches (5 cm) in length, about 1 inch to 2 inches (2.5-5 cm) in width, and about ½ ″inch (12 mm) in thickness. The bars 509 can fit between an OR table rail and the OR table proper. These bars can stabilize the attachment of the table 500 or can be configured to secure the table 500 to the OR table. Alternatively, clamps or other mechanical attachment mechanism can be used to secure the table 500 to the OR table.

As shown in FIGS. 11A and 11B, the table 500 includes a platform 501 that includes a substantially planar top surface 502 and a bottom support surface 506. The platform 501 defines a recess 504 formed therein. The recess 504 is sized and configured to locate an irrigation platform on the platform such that a work surface of the irrigation platform (see, for example, support platform 14 of FIG. 1) is substantially level with the top surface 502. The bottom support surface 506 can be a relatively thin plastic material configured to support the irrigation platform and at least a portion of the extremity.

The recess 504 can be sized to hold the irrigation platform securely in place in at least one direction or can be sized to allow sliding movement of the irrigation platform. For example, the recess 504 can be sized such that the irrigation platform can move relative to the operating table; e.g., toward/away from the operating table, along a length of the operating table, or both. The relative movement of the irrigation platform accommodates varying lengths of extremities and the repositioning of the platform as necessary during the procedure. In one embodiment, the recess 504 has a generally rectangular shape and measures about 9 inches to about 12 inches (22.5-30 cm) in width and about 18 inches to about 24 inches (45-60 cm) in length and extends to the end of the table 500 opposite the proximal end 508.

The table 500 can include one or more pedestals or support legs 510. In one embodiment, the support leg 510 is located in a proximal area of the table 500. This arrangement allows easy positioning of an X-ray machine (for example, the arm of a C-arm X-ray unit that has to be located under the table 500) along with the seating of the surgeon and assistants. The support leg 510 can be attached to the underside of the table 500 by a pivot type connection 511 that allows the support leg 510 to be folded relative to the table 500 for storage or in the event that the table 500 is supported by the OR table (see, for example, FIG. 11D). The support leg 510 can also be connected to the table 500 via a hinge 514. Various types of hinges can be used, for example, a table leg hinge, a folding leg support hinge, or a barrel type hinge. The support leg 510 can be secured to the underside of the table 500 when not in use. In addition, the height of the support leg 510 itself is adjustable.

As shown in FIGS. 11B and 11C, the support leg 510 has two pieces, an upper support leg or housing 510b and a lower leg 510a. In one embodiment, the housing 510b is attached to the underside of the table 500 about 8 inches to about 12 inches (20-30 cm) from the proximal end 508 of the table 500 and equidistant from each side of the table 500. When two legs 510 are provided, the legs 510 can be spaced about 4 inches to about 20 inches apart (10-50 cm) and equidistant from each side of the table 500. The leg 510 includes a shallow U-shaped rim with the open portion of the U directed away from the proximal end 508 of the table 500. The body of the housing 510b is pivotally coupled to the U-shaped rim such that the support leg 510, when in use, rests against the table 500 inside the rim. When folded, the support leg 510 rests against the open portion of the U and lies along the underside of the table 500. It is desirable to manufacture the rim from a metal or hard plastic material to withstand the repeated stresses at this point. Additionally, a collar can also be included to further stabilize the connection of the leg 510 to the table 500.

The upper leg portion of the housing 510b is about 3 inches to about 6 inches (7.5-15 cm) in length and about 2 inches to about 4 inches (5-10 cm) in width. The housing 510b can be made from a piece of square tubing. Disposed along the length of the housing 510b on the sides parallel to the outer sides of the table 500 are a series of transverse holes 513 spaced about 1 inch (2.5 cm) apart. The holes can have a diameter of about ¼ to ½ inch (6-12 cm). The side of the housing 510b facing away from the proximal end 508 of the table 500 is coupled to one end of the afore-mentioned hinge 514. The other end of the hinge 514 is attached to the table 500 distal to the open portion of the rim.

The lower leg 510a can be about 28 inches to about 32 inches (70-80 cm) long and its cross-sectional dimension will be sized to slidably fit within the upper leg 510b. The lower leg 510a can include a foot 516 that rests against the floor and expands in cross-section to about 4 inches to about 6 inches (10-15 cm). The lower leg 510 also includes a series of transverse holes 513 disposed on an upper portion thereof that are sized and located to correspond with the holes in the housing 510b (i.e., spaced 1 inch (2.5 cm) apart and ¼ to ½ inch (6-12 mm) in diameter). The support leg 510 also includes a peg 512 that measures about ¼ to ½ inch (6-12 mm) in diameter and about 2½ inches to about 4½ inches (6-11 cm) in length and is fastened by a chord 517 to the housing 510b. The peg 512 can be made from a metal or other suitably strong material and can be easily removed and passed through the holes 513 in the two leg portions 510a, 510b to secure the leg portions relative to one another. The peg 512 and hole 513 arrangement is also used to facilitate adjustment of the table's height by sliding the leg portions 510a, 510b relative to one another with the peg 512 removed, until the holes 513 are aligned and the peg 512 inserted.

In an alternative embodiment, the upper and lower legs 510a, 510b can be threadedly engaged, either directly or through the use of a threaded collar or other screw-type mechanism. This arrangement allows the height of the table 500 to be adjusted by rotating one or both of the legs 510a, 510b. Alternatively, the leg 510 can include a pneumatic mechanism for adjusting the height of the table 500.

FIG. 12 depicts an irrigation and retraction platform 410 in accordance with one embodiment of the invention, with a patient's extremity secured thereon. A hand 402 is positioned on the platform 410 and secured in place with various restraints 450, 470. The platform 410 and restraints 450, 470 are in accordance with those described hereinabove.

As shown, four finger restraints 450A are inserted through a first set of longitudinal openings 416A to secure the fingers in place. A thumb restraint 470 is used to hold the thumb away from the palm of the hand 402. A projection on the thumb restraint 470 securely engages one of the holes 42 on the platform. One wrist restraint 450B is inserted through a second set of longitudinal openings 416B to secure the distal forearm in place and one forearm restraint 450C is inserted through a third set of longitudinal openings 416C to secure the proximal forearm in place. More or fewer restraints 450 can be used to restrain the extremity depending on the nature of the procedure and the extremity to be operated on.

The sizes and shapes of the platforms, tables, and enclosures will vary to suit a particular application (e.g., hand surgery or leg surgery) and patient (e.g., large adult, small adult, or pediatric). In addition, the dimensions given herein are exemplary only and will be determined as necessary for a particular application.

The various components described herein can be manufactured from any suitable medical grade polymeric or metal materials or combinations thereof. The polymeric materials can include reinforcement. Suitable polymeric materials include: polyurethanes, such as a thermoplastic polyurethane (TPU); ethylene vinyl acetate (EVA); thermoplastic polyether block amides, such as the Pebax® brand sold by Elf Atochem; thermoplastic polyester elastomers, such as the Hytrel® brand sold by DuPont; thermoplastic elastomers, such as the Santoprene® brand sold by Advanced Elastomer Systems, L.P.; thermoplastic olefin; nylons, such as nylon 12, which may include 10 to 30 percent or more glass fiber reinforcement; silicones; polyethylenes; acetal; and equivalent materials. Reinforcement, if used, may be by inclusion of glass or carbon graphite fibers or para-aramid fibers, such as the Kevlar® brand sold by DuPont, or other similar method. Also, the polymeric materials may be used in combination with other materials, for example natural or synthetic rubber. Metal materials include stainless steel, carbon steel, and aluminum. Other suitable materials will be apparent to those skilled in the art. Generally, the materials used should be capable of sterilization.

The various polymeric components can be manufactured by, for example, injection molding or extrusion. Extrusion processes may be used to provide uniform shapes, such as a single monolithic frame. Insert molding can be used to provide the desired geometry of, for example, the openings. Additional openings and features can be created in the desired locations by subsequent machining operations. Other manufacturing techniques include melting or bonding additional portions, for example, with a liquid epoxy or a hot melt adhesive, such as EVA. In addition to adhesive bonding, portions can be solvent bonded. The various components can be separately formed and subsequently attached. The metal components can be manufactured by conventional machining and forming processes, as known to those of skill in the art. In some embodiments, various components are commonly available hardware items that can be purchased and mechanically attached to various other components.

Having described certain embodiments of the invention, it will be apparent to those of ordinary skill in the art that other embodiments incorporating the concepts disclosed herein may be used without departing from the spirit and scope of the invention. The described embodiments are to be considered in all respects as only illustrative and not restrictive.

What is claimed is:

1. An apparatus for supporting and restraining an extremity of a mammal during surgery, the apparatus comprising:
a chamber comprising a bottom surface and four side walls;
an opening defined by at least one of the bottom surface and sidewalls;
a support platform disposed above the bottom surface and configured to engage at least a portion of the chamber; and
a plurality of longitudinal perforations disposed through the support platform and arranged across the support platform, wherein the longitudinal perforations vary in size.

2. The apparatus of claim 1, further comprising a plurality of restraints for interacting with pairs of correspondingly sized longitudinal perforations.

3. The apparatus of claim 1, wherein the plurality of longitudinal perforations comprises a first portion of perforations arranged in a distal portion of the platform, the first portion of perforations oriented and sized to receive multiple finger restraints.

4. The apparatus of claim 3, wherein the first portion of perforations are arranged in a radial pattern.

5. The apparatus of claim 3, wherein the first portion of perforations are sized and arranged to provide multiple points of restraint for each finger.

6. The apparatus of claim 1, wherein the plurality of longitudinal perforations comprises a second portion of perforations arranged in a medial portion of the platform, the second portion of perforations oriented and sized to receive at least one wrist restraint.

7. The apparatus of claim 1, wherein the plurality of longitudinal perforations comprises a third portion of perforations arranged in a proximal portion of the platform, the third portion of perforations oriented and sized to receive at least one forearm restraint.

8. (canceled)

9. The apparatus of claim 1 further comprising a mesh layer disposed on the support platform and covering at least a portion of the plurality of perforations.

10. The apparatus of claim 1 further comprising at least one suture retraction peg disposed along a periphery of at least one of the chamber and the support platform.

11. The apparatus of claim 1 further comprising a positioning pad releasably disposable on the support platform for supporting flexion of a portion of the extremity.

12-18. (canceled)

19. The apparatus of claim 1, wherein the plurality of restraints comprise:
a first set of restraints sized for variably securing a finger; and
at least one second restraint sized for variably securing a wrist.

20. The apparatus of claim 1, wherein each restraint comprises:
an elongate strap biased into a planar orientation;
a plurality of projections arranged on a first end of the strap; and
a second corresponding plurality of projections arranged on a second end of the strap.

21. The apparatus of claim 1 further comprising a gel pad disposed over at least a portion of the support platform.

22-23. (canceled)

24. The apparatus of claim 1, wherein the support platform comprises a plurality of holes sized and arranged to receive at least one thumb restraint.

25-27. (canceled)

28. The apparatus of claim 1 further comprising an enclosure configured for sealingly engaging at least one of the chamber and the support platform.

29. The apparatus of claim 28, wherein the enclosure comprises:
a first opening disposed on one end of the enclosure and configured for receiving at least a portion of a patient's extremity; and
at least one second opening disposed on a side of the enclosure and configured for receiving at least one of a hand, a wrist, a forearm, and a surgical instrument.

30-31. (canceled)

32. An apparatus for assisting in performing a surgical procedure on an extremity of a patient, the apparatus comprising:
a support platform for at least one of supporting and restraining at least a portion of the extremity; and
an enclosure configured to sealingly engage the support platform, the enclosure comprising:
a first opening disposed on one end of the enclosure and configured for receiving at least a portion of the extremity; and
at least one second opening disposed on a side of the enclosure and configured for receiving at least one of a hand, a wrist, a forearm, and a surgical instrument.

33. The apparatus of claim 32, wherein the enclosure further comprises a port disposed on a top surface thereof and configured for receiving a surgical instrument.

34. A surgical table comprising:
a platform comprising a substantially planar top surface and defining a recess configured to receive an irrigation platform, the recess sized and configured to position a work surface of the irrigation platform substantially level with the top surface; and
a mechanism for securing the platform to an operating table.

35. The surgical table of claim 34, wherein the recess is sized and configured to allow horizontal positioning of the irrigation platform relative to the operating table.

36-38. (canceled)
